(19) United States
(12) Patent Application Publication
(10) Pub. No.: US 2024/0145096 A1
(43) Pub. Date: May 2, 2024

(54) DIGITAL SYSTEM FOR AUTOMATED MEASURING OF RELATIVE RISK MEASURANDS AND SCORES OF LIVING INDIVIDUALS AND METHOD THEREOF

(71) Applicant: Swiss Reinsurance Company Ltd., Zürich (CH)

(72) Inventors: Douglas RIX, Zürich (CH); John SCHOONBEE, Zürich (CH); Michael DUCKER, Zürich (CH); Alan MARTIN, Zürich (CH); Aspasia ANGELAKOPOULOU, Zürich (CH)

(73) Assignee: Swiss Reinsurance Company Ltd., Zürich (CH)

(21) Appl. No.: 18/373,566

(22) Filed: Sep. 27, 2023

Related U.S. Application Data
(63) Continuation of application No. PCT/EP2022/067968, filed on Jun. 29, 2022.

(30) Foreign Application Priority Data
Jun. 29, 2021 (CH) 00755/21

(51) Int. Cl. G16H 50/30; G06Q 40/08; G16H 50/20; G16H 50/70

(52) U.S. Cl. CPC G16H 50/30; G06Q 40/08; G16H 50/20; G16H 50/70

(57) ABSTRACT

Proposed is a system and method for measuring relative risk measurands of living individuals. The relative risk measurands provides a measure for the frequency of occurrences of specific medical events having impacting consequences in specified ranges to the living individual within a defined cohort of living individuals relative to a randomized cohort of living individuals. The system provides automation of the risk assessment and measuring process with a high efficiency and accuracy.

### CROSS REFERENCE TO RELATED APPLICATIONS

The present application is a continuation application of International Patent Application No. PCT/EP2022/067968, filed Jun. 29, 2022, which is based upon and claims the benefits of priority to Swiss Application No. 00755/21, filed Jun. 29, 2021. The entire contents of all of the above applications are incorporated herein by reference.

### FIELD OF THE INVENTION

The present invention relates to systems and methods in the field of automation of life/health risk assessment and measuring. In particular, the invention relates to automated systems for measuring relative risk measurands of living individuals, where the relative risk measurands provide a measure for the frequency of occurrences of specific medical events having impacting consequences in specified ranges to the living individual within a defined cohort of living individuals relative to a randomized cohort of living individuals. Further, the present invention relates to digital system, in particular risk-transfer systems and related technology, relying on automated risk assessment as a crucial element in life risk-transfer technology to classify the applicants e.g. for automated underwriting processes.

### BACKGROUND OF THE INVENTION

Automation in risk assessment and measurement is a crucial element in the life risk-transfer technology to allow automated classification of applicants of risk-transfers or to allow automated optimization of the risk cover of applicants or to automated detection of gabs in the risk cover of an applicant or portfolios of applicants. Risk-transfer systems or risk-transfer providers perform underwriting process to make decisions on applications and to price policies accordingly. With the increase in the amount of data and advances in data processing techniques, the underwriting process can be automated for faster processing of applications. In the last decade, the big data technologies revolutionize the way risk-transfer systems are enabled to collect, process, analyze, and manage data more efficiently. Thus, automation technology proliferates in various sectors of risk-transfer technology such as risk assessment, customer analytics, product development, automated marketing analytics, claims analysis, underwriting assessment and processing, fraud detection, and multi-layer risk-transfer automation. Telematics is a typical example where big data analytics is being vastly implemented and is transforming the way auto risk-transfer pricing the premiums of individual drivers. Individual life and health insurance organizations still rely on the conventional, not-automated actuarial formulas to predict mortality rates and premiums of life policies. Life risk-transfer technology has however started carrying out automated predictive data-processing to improve the operational efficacy of the automation, but there is still a lack of efficient and optimized automation operating on a high technical accuracy. Many prior art solution have been concentrated on data mining techniques to detect frauds among risk-transfer systems, which is a crucial issue due to the problem facing great losses.

In the prior art systems, measurable risk parameter values associated with a specific, typically modifiable life-style pattern are known. They comprise e.g. heavy alcohol drinking, smoking, excess body weight and lack of physical exercise as modifiable risk factors of lifestyle, which may all contribute to the incidence of chronic diseases and premature death. There may also be synergistic and additive interactions between such factors in individuals with clustering of unfavorable lifestyle factors. Therefore, interventions aimed at reducing the number of risk factors has been recognized as an important target in both personalized medicine and public health policies. Recent studies have estimated that adopting a healthy lifestyle even at the age of 50 could add more than a decade to life suggesting significant therapeutic potential for lifestyle interventions.

Measuring parameters such as increased gamma-glutamyltransferase (GGT), and alanine aminotransferase (ALT) enzyme activities in apparently healthy individuals may be attributed to unhealthy lifestyle factors, such as alcohol consumption or excess body weight. The increases in these liver enzymes may also associate with extra-hepatic disease risks, including metabolic syndrome, and cardio- or cerebrovascular events. While the biochemical pathways underlying such observations often remains unclear, it can be assumed that inflammatory processes, oxidative stress and generation of abnormal lipid profiles are key pathogenic factors in the sequence of events leading to hepatotoxicity or other adverse health effects, such as incident stroke, in individuals presenting with various clusters of risk factors.

So far, it is still complex to examine the individual and joint impacts of the various unfavorable life style factors on biochemical indices of health. In particular, it is technically challenging to measure the combined effects of various lifestyle-related factors on biomarkers of liver status (ALT, GGT), inflammation (C-reactive protein) and lipid metabolism (cholesterol, HDL-cholesterol, LDL-cholesterol, triglycerides) in a large population, which includes detailed records on alcohol consumption, smoking, physical activity and health status. It can be assumed that precise measurements of the biomarker behavior in response to various types of unhealthy behaviors may improve the possibilities for automated interventions and alarm signaling aimed at adopting more favorable lifestyles and adapt a more favorable risk score measure.

There is a need to develop measurements on health and lifestyle which can be, inter alia, validated for use in international population-based health measurements. The measured parameter value on each parameter measured, such as alcohol consumption, smoking, physical activity and coffee consumption should preferably be chosen to be assigned to mutually exclusive and collectively exhaustive categories. Data on alcohol consumption can e.g. be measured from the past 12 months prior to blood sampling and included information on the types of beverages consumed as well as the amounts and frequencies of consumption. The ethanol content in different beverages can e.g. be quantitated in grams of ethanol based on defined portion sizes or threshold values, for example, as follows: regular beer 12 grams (⅓ L), strong beer 15.5 grams (⅓ L), long drink 15.5 grams (⅓ L), spirit 12 grams (4 cL), wine 12 grams (12 cL) and cider 12 grams (⅓ L). Information on smoking habits was collected with a set of standardized questions and the data was expressed as the amounts of cigarettes per day. Habitual physical activity including both the number and total time used for physical exercises can also be measured from an uer. Coffee consumption was assessed with a set of standardized questions and expressed as the intake of standard servings of coffee (cups) per day.

The measuring values obtained from measuring can e.g. subsequently be used to define scores index measure, for example, for low risk (=0), medium risk (=1) and high risk (=2) categories for each individual risk factor following captured patterns on health-related risk assessment in relation to alcohol consumption, smoking, BMI status and physical activity. Herein, the variables can e.g. be categorized into three ordinal levels to yield increased statistical power as compared to previously used prior art dichotomous classification. For alcohol consumption the score measures can e.g. be defined as follows: 0=no consumption; 1=alcohol consumption between 1-14 (men) or 1-7 (women) standard drinks per week; 2=alcohol consumption exceeding 14 drinks (men) or 7 drinks (women) per week. For smoking 0=no smoking, 1=1-19 cigarettes per day, 2=≥20 cigarettes per day; for BMI 0=BMI<25; 1=BMI≥25 and <30; 2=BMI≥30. For physical activity 0 represents those with physical activity over 4 hours per week; 1=those with physical activity between 0.5 and 4 hours per week and 2=those with physical activity less than 30 min/week. The sum of these score measures can e.g. be used to measure a total number of risk factors, with higher score measures (e.g. maximum=8) indicating an unhealthier lifestyle. Serum liver enzymes (ALT and GGT) can e.g. be measured using a standard clinical chemical methods, such as on an Abbott Architect clinical chemistry analyzer of the manufacturer (Abbott Laboratories, Abbott Park, Ill., USA). High-sensitivity CRP, a biomarker of inflammation, can e.g. be determined using a latex immunoassay (Sentinel Diagnostics, Milan, Italy) with the Abbott Architect c8000 clinical chemistry analyzer. Lipid profiles can e.g. include determinations of total cholesterol, high-density lipoprotein-associated cholesterol (HDL), low-density lipoprotein (LDL) and total triglycerides using standard enzymatic methods. All laboratory or telematics-based measurements can be subjects to cut-offs for the normal limits of the different markers e.g. as follows: ALT (50 U/L men; 35 U/L women), GGT (60 U/L men; 40 U/L women), CRP (3.0 mg/L), cholesterol (5 mmol/L), HDL cholesterol (1.0 mmol/L men, 1.2 mmol/L women), LDL cholesterol (3.0 mmol/L), triglycerides (1.7 mmol/L).

The characteristics can e.g. be measured using pattern recognition, machine-learning or applied analysis structures of variance (ANOVA) e.g. with polynomial contrasts to reveal possible trends across increasing risk score categories. The distribution of abnormal biomarker levels across the risk categories can e.g. be analyzed by chi-square measurements for trend. Binary logistic regression can e.g. be used to estimate the odds ratios (ORs) of abnormal biomarker levels associated with the risk score categories, adjusting for age and coffee consumption, as these factors are known to potentially associate with abnormal biomarker levels and showed association in univariate analysis. All factors can e.g. be inputted simultaneously into the multivariable modelling and/or machine-learning structure. Potential multicollinearity among the covariates can e.g. be measured by generating the Variance Inflation Factor (VIF). Correlations between the risk scores and various biomarkers can be measured using Spearman's rank correlation coefficients. For significance, a defined threshold p-value can be implemented e.g. considering p-value <0.05 statistically significant.

Further in the prior art, automated or semi-automated risk-transfer systems, typically interacting with a user via graphical user interface (GUI), are known. In particular, automated, cloud-based systems enabling an end-user (e.g. an end-user being a consumer or an employee of a business as an insurance or distribution company (e.g. broker)) to compose automatically a first-tier (insurance) and/or second-tier (reinsurance) risk-transfer product, after conducting a dialogue with a knowledge-based system, are known. Such systems reduce the dependences of first-insurers or reinsurers on both their information technology (IT) and their human experts, as e.g. actuarial experts. Such systems are able to adjust the dialogue interactively according to the specific needs of the users and ask for the relevant data needed for the desired risk-transfer product. However, vendor-specific digital retail stores for providing automated or semi-automated risk-transfer do not allow to aggregate, compare and monitor products from a wide array of provider systems as e.g. carrier or broker units. Further, selection is usually narrow, and availability is low.

There are different categories of digital risk-transfer platforms, in particular so called digital active and passive risk-transfer management platforms and digital marketplaces, which intend to provide aggregating digital available products from a wider array of providers. There, selection is technically wider, and availability is higher than in vendor-specific digital retail stores. A digital marketplace is a type of electronic platform where product or service information is provided by multiple third parties. Online marketplaces are the primary type of multichannel electronic commerce and can be a way to streamline the automated production process. In a digital marketplace, user transactions (i.e. interaction between carrier/broker and carrier/broker in B2B interaction (insurer/distributor driven) or interaction between carrier/broker and consumer in B2C interaction) are automatically processed by the digital marketplace platform and then made accessible to and completed by the participating retailer or wholesaler systems. These type of digital systems allow user systems to register, e.g. as client systems, thereby linking and transacting single items to many items e.g. for a “post-selling” fee. Some of the prior art digital marketplaces provide automated business-to-business (B2B) trading. Such examples of digital platforms that enabled electronic commerce between clients include e.g. VerticalNet, Commerce One, Covisint, or Aladdin by Blackrock. Other B2B online marketplaces focus on a limited range of digital service, such as EC21, Elance and eBay, and have not achieved the dominance digital marketplaces have obtained in B2C retail. B2B purchasing requires that digital marketplaces facilitate and automate complex digital transactions, such as a request for quotation (RFQ), a request for information (RFI) or request for proposal (RFP). Digital marketplaces belong to the technical field of information technology acting as digital intermediaries by connecting buyer systems and seller systems. There are digital marketplaces for the online outsourcing of services like IT services, search engine optimization, marketing, and skilled crafts and trades work. Microlabor digital marketplaces such as Upwork and Amazon Mechanical Turk allow freelancers to perform tasks which only require a client device, e.g. a computer device, and internet access, i.e. access to the world-wide backbone network. For example, Amazon's Mechanical Turk digital marketplace focuses on “human intelligence tasks” that are difficult to automate computationally and/or by other technical means. This includes so called content labelling and content moderation.

Typically, for many types of risk-transfer, the structure of the risk-transfer concerned makes them suitable for automation with regard to the automated composition/configuration of the risk-transfer products, the so-called UnderWriting (UW). Most risk-transfers consist of a number of common basic parts and elements to configure. These parts and elements are herein referred as “structuring blocks” of the risk-transfer, i.e. the risk-transfer structure defining the characteristics of the risk-transfer. Different combinations of these structuring blocks lead to different risk-transfer products. For a human-machine interface (HMI) as e.g. Graphical User Interface (GUI), and for the corresponding necessary dialogue between the machine and the user, it should be possible to automatically compose risk-transfer products or structures out of a set of such structuring blocks. The “human” is the user of the automated system, i.e. an insured or an insurer depending if a first-tier (insurance product) or second-tier (reinsurance product) risk-transfer has to be configurated, while the “machine” refers to the automated system, as e.g. an automated web server or cloud-based system or digital system of a provider e.g. an insurer for first-tier risk-transfer products or a reinsurer for second-tier risk-transfer products. For example, using the parameterization of structuring blocks, typically a system comprising a limited amount of structuring blocks can be obtained for non-customized risk-transfer products. In an example of life risk transfer, applying domain knowledge of the actuary filed of the life risk-transfer structures (structure given by the insurance product parameters), the risk-transfer structure (defined by the policy parameters) comprises the steps of defining and transferring a monetary amount transfer (premium) either on basis of a regular monetary transfer or as transfer of a lump sum, the user (beneficiary) receiving an endowment if the insured is alive, which means that the insurer has to pay the beneficiary a sum of money (i.e. the benefit) in the case of death in exchange for the premium transfer. Depending on the risk-transfer parameters, other events such as terminal illness or critical illness can also trigger payment. Unseen the latter, the life risk-transfer can therefore be reduced to three basic event blocks. These events can be assigned to a set of structuring blocks common to all life risk-transfer: premium endowment and alive. For automation, all such systems depend on triggers. Triggers detect events in its environment by observing, measuring and/or monitoring properties or characteristics of input stimuli, as measuring parameters, it receives or measures. Finally, based on the used structuring blocks, the dialogue input flow of the user interface (HMI) may follow pairs of current state and input to what the present output and the next state must be. Thereby, the input triggers the next state.

Today, automation of the underwriting process is not enough to cope with the challenges. Risk-transfer, at the level of the first tier risk transfer (insurance) or second tier risk transfer can e.g. be broader supported by diversifying the risk shares of the risk transfer. However, the automated, machine-based quantifying and optimizing the risk measure by varying the risk shares is not a part of normal UW processes. Further, the increasingly dynamic and diversified (re)insurance market requires shorter time-to-market of highly customized (re)insurance products. Such process are technically difficult to automatize. Though prior art systems are able to automate or semi-automate the underwriting process, there is still a need for more completely automated electronic solutions covering the whole risk-transfer. In particular, there is no system providing a fast, consistent and easy access to reinsurance risk-transfer, thereby allowing to reduce administration costs for managing risk portfolios, (ii) to access fast, automatic capacity approval for medium-sized single risks or facilities, and (iii) to relieve administration time, to focus on more complex parts of the risk-transfer. In summary, there is a need for more efficient digital risk placement, claim handling and accounting channels for users covering possibly the whole process of the risk-transfer, i.e. the entire value chain providing an end-to-end process, thereby providing fast composing, launch and configuration of highly customizable (re)insurance products.

Another subject is operational stability and endurance of risk-transfer system in the varying external condition of the market. Conventionally, there are different broad technical categories of automated risk-transfer portfolio management systems. As mentioned, one prior art risk-transfer portfolio management system category is based on active management, wherein the risk-transfers are selected by the system for a portfolio individually based on measures quantifying impacting economic, financial, credit, and/or business parameters, based on technical trends, based on cyclical patterns etc. Another conventional category is passive management systems, also called indexing, wherein the risk-transfers in a portfolio duplicate those that are triggered by an index measure. The risk-transfers, in a passively managed portfolio, are weighted by relative market weighting or equal weighting (e.g. based on capitalization parameters). Another middle ground conventional category of risk-transfer portfolio management systems are called enhanced indexing systems, in which a portfolio's characteristics, performance and holdings are substantially dominated by the characteristics, performance and holdings of the index measure, albeit with modest active management departures from the index measure. The risk-transfer or (re)insurance marketplace consists of numerous products that have evolved out of the most basic designations of risk exposure and accumulation of risk balancing resources. Methodologies for scoring and rating the products that are associated with differences given by the operational parameters can result in real price and term discontinuities. As known, it is possible through one kind of risk-transfer product engineering to change high risk-transfer portfolios into lower risk ones through various kinds of aggregation, diversification, hedging and division of risk. As a result, a single C risk can be reconfigured into a product that appears to have AAA-risk characteristics. Of course, the individual risk-transfer product retains the same characteristic that it always had; it is the pooled, reconfigured and reengineered aggregation of the risk-transfers that is measured differently. The difference is most easily seen as the portfolio becomes increasingly granular. The disease in the form of a potentially toxic alignment of risk elements that might occur in either a singular or complex alignment of risks that might infect one class or sub-class of risk-transfer products may not necessarily spread to the whole portfolio or it, in contrast, generate a systemic risk. Risk, as used herein, is defined as a technical term, i.e. as a measurable probability measure for an occurrence or a plurality of occurrences of a defined, physical event having a measurable impact on a risk-exposed object or individual, wherein the risk measure relates to a predefined time-window (present of future) and geographic location.

A measurable quantity of the stability and endurance of an automated system, i.e. an automated (re)insurance unit, can be provided by a quantified resilience measure. Resilience is a measure for the operational stability and ability of staying power of a system through changing market parameters, market peaks and valleys, across established and emerging markets, and spanning decades of years in the market. It is also measure for the system's ability to attract new customers, and keep customers loyal to the use of the system in the market. It is to be noted, that for the measured quantity of the present invention, the measured resilience score is defined as introduced below.

Finally, a further disadvantage of the known prior art digital platforms and marketplaces is that those automated structures are not able to build up a complete automation technology (e.g. due to missing technical solution providing efficient and optimized risk assessment based on reliable input measuring parameters) providing a digital environment for supplier systems, e.g. insurer units, broker units, agent units etc. and consumer units, e.g. insured, where all units are able to participate in automated, digital processed transactions and risk-transfers covering B2B (Business to Business) as well as B2C (Business to Consumer) structures. B2B and B2C structures are based on two very different models, requiring for automation different skills, disciplines and mind-sets. In the prior art, the systems typically try to avoid running with both without splitting the system. B2C is where there is a buyer unit and a seller unit and they interact electronically at the digital platform as a point of sale or point of transaction. The point of sale could be realized as a digital marketplace or cloud-based platform. At the point of sale, the technical system provides an interaction between the units. In B2B there is no structure which can be denoted as a “point of sale”. In digital systems based on B2B interaction, there is a relationship or linkage. Typically, it is a somehow developing or evolving relationship. Thus, a point of sale is B2C and a long-term relationship or linkage is B2B, both having different technical issues such as the automation of the Key Account Management and Customer-Relationship-Management CRM (B2B), and the automation of the Net Promoter Score and Customer-Experience-Management CXM (B2C) become easier to understand. For example, customer satisfaction surveys can be a solution for B2B relationship audits, but are inappropriate for B2C, since the essence of B2B are strong relationships, solutions to problems joint-ventures designing new warehouses, joint development and testing of new products. In the example of insurance systems, most insurance system activity can be classed as B2C, including risk-transfers for motor, house and small business units. However, property developers, ship owners and big businesses have specialist needs and typically will involve human experts as risk managers to liaise with their insurance companies in order to keep the risk-transfer cover and the risk exposure at appropriate levels at all times. This is where B2B structures provide appropriate relationships. Insurance systems will have dedicated units to look after these customers. In other words, the B2C relationship between customer, broker and insurer is linear whereas the specialist B2B relationship between customer, broker and insurer is triangular or circular.

Concerning the automation of digital platforms and digital driven marketplace platforms, machines and automated agents are increasingly involved in digital market activities, including for data collection and measuring, forecasting, planning, transaction execution, and other activities. This includes increasingly high-performance systems, such as used in high-speed trading. A need exists for methods and systems that improve the machines that enable markets, including for increased efficiency, speed, reliability, and the like for participants in such markets. Many digital marketplace platforms are increasingly distributed, rather than centralized, with distributed ledgers like Blockchain, peer-to-peer interaction models, and micro-transactions replacing or complementing traditional models that involve centralized authorities or intermediaries. A need exists for improved machines that enable distributed transactions to occur at scale among large numbers of participants, including human participants and automated agents. Operations on blockchains, such as ones using cryptocurrency, increasingly require energy-intensive computing operations, such as calculating very large hash functions on growing chains of blocks. Systems using proof-of-work, proof-of-stake, and the like have led to “mining” operations by which computer processing power is applied at a large scale in order to perform calculations that support collective trust in transactions that are recorded in blockchains. Many applications of artificial intelligence also require energy-intensive computing operations, such as where very large neural networks, with very large numbers of interconnections, perform operations on large numbers of inputs to produce one or more outputs, such as a prediction, classification, optimization, control output, or the like. A major challenge for human life risks and asset owners' risk and carriers of risk-transfer is the uncertainty involved in optimizing a living object or asset in respect to its measured risk exposure, or also associated risks such as resulting from volatility in the cost and availability of improvements (in particular where less stable renewable resources are involved), variability in the cost and availability of resources, and volatility and uncertainty in various end markets to which risk-improving or risk-protecting resources can be applied, among other factors.

The document US 2018/218453 A1 teaches a system for automated, autonomous management of risk transfers. The system provides a more functional approach to automation of a risk-transfer system, while providing fraud detection capabilities to prevent abuse of the system. The system analyzes available data to determine contracts and offerings that are acceptable for both the risk-exposed requester for the risk-transfer and the risk-transfer (providing) system by balancing the portfolio of the risk-transfer system. The system also provides a process claim structure, and automated managing structure to automatically generate the payout for approved claims. The system comprises a network-connected server with an interface for a requester to submit a request. An automated underwriting processor generates contract blocks by compiling the request into a computational graph-based format, linking the contract block to the requester, storing the contract block into memory, retrieving a plurality of available underwriting agreements from memory, and creating an offer list by perform computational graph operations on the contract block to determine viable risk-transfer agreements. Finally, the system presents the offer list to the requester via the interface.

### SUMMARY OF THE INVENTION

It is an object of the invention to provide an automated optimized risk assessment and risk measuring system, operatable in the field of life and health risks, thereby providing the technical basis for automated risk assessment and risk-transfer (including automated underwriting (UW)). The system should allow for systematic capturing, measuring, quantifying, and forward-looking generating of appropriate risk and risk accumulation measures of risk-transfers and risk-transfer portfolios associated with risk exposures of living individuals (humans) based on physical measuring parameter values and data, i.e. the impact of a possibly occurring physical event in a defined future time window. In the present invention, this includes measuring and rating the score measures (“resilience score”) of the life or health of a person. It is a further object of the present invention to propose a processor-driven system or platform providing an automated digital channel for automatically concluding and dynamically adapting risk-transfers between a risk-transfer service user and a risk-transfer service provider, which does not exhibit the disadvantages of the known systems. In particular, it is an object of the present invention to provide an inventive technical teaching for automation which is easily integratable in other processes, productions chains or risk assessment and measuring systems, e.g. by appropriate APIs. This invention also aims at providing an automated system to enhance risk assessment of individuals as applicants for a risk-transfer or among life risk-transfer systems using predictive processing efficiency and accuracy.

According to the present invention, these objects are achieved particularly through the features of the independent claims. In addition, further advantageous embodiments follow from the dependent claims and the description.

According to the present invention, the above-mentioned objects are particularly achieved by the inventive, automated, digital measuring system for measuring relative occurrence frequencies or more specifically, a measurable occurrence probability of a medical event and/or health event and/or life risk event (given by as a measuring value of risk measurands or score index measurands) of living individuals for a future measurement time window, wherein the relative risk measurands provide a measure for the frequency of occurrences of specific medical events having impacting consequences in specified ranges to the living individual within a defined cohort of living individuals relative to a randomized cohort of living individuals, in that the system comprises a defined parametrization for capturing risk shape pattern for living individual, the parametrization of the risk shape pattern comprising at least lifestyle factor values measuring physical activity and/or sleep and/or nutrition and/or mental wellbeing and/or substance use and/or environmental conditions, in that the system captures a multitude of risk shape pattern for living individuals at least by means of said lifestyle factor values, wherein the captured multitude of risk shape pattern are clustered by the system, each cluster defining a prototype of risk shape pattern assigned to a dedicated relative risk measurand, in that a newly captured risk shape pattern of a living individual is mapped by the system to one of the prototypes of risk shape pattern based on measured lifestyle factor values associated with the living individual, wherein the dedicated relative risk measurand assigned to the risk shape pattern is outputted as resilience score value of the living individual. The invention has inter alia the advantage that it provides an increasingly optimized risk assessment improving the technical accuracy, flexibility and transparency of such automated system. Further, the system allows to provide and dynamically adapt clustering and rating based on prototypes of risk shapes, and thus providing an automated underwriting risk measurement and rating with high technical accuracy. The invention also has, inter alia, the advantage that the system can be modified or adapted to produce relevant individual (consumer) facing scores to match a specific development in a given market (e.g. relative risk rating, health age etc.). The inventive system also has the advantage, that it allows a dimensionality reduction to rely on relevant measuring parameters that can improve the prediction power of the modeling structure without having redundancies. The data dimension can e.g. be reduced by the system itself, using integrated feature selection techniques and feature extraction namely, Correlation-Based Feature Selection (CFS) and Principal Components Analysis (PCA). Machine learning algorithms, namely Multiple Linear Regression, Artificial Neural Network, REPTree and Random Tree classifiers were implemented on the dataset to predict the risk level of applicants.

In an embodiment variant, the parametrization of the risk shape pattern further comprises clinical factor values measuring (i) build factor values comprising a measured height and/or weight factor value, and/or (ii) lipids factor values comprising a measured total cholesterol factor value and/or a high-density lipoprotein factor value and/or a triglycerides factor value, and/or (iii) blood pressure factor values comprising a measured systolic and diastolic blood pressure factor value, and/or (iv) glucose metabolism factor values comprising a measured fasting/non-fasting glucose factor value and/or glycated hemoglobin (hemoglobin A1c) factor value and/or diabetes status, and/or (v) liver function factor values comprising a measured gamma-glutamyltransferase (GGT) factor value and/or an alanine transaminase (ALT) factor value and/or an aspartate transaminase (AST) factor value and/or an alkaline phosphatase (Alk Phos), and/or (vi) family history of diabetes and circulatory disorders factor values. The parametrization of the risk shape pattern can e.g. further comprise clinical factor values measuring (i) a measured calcium score factor value, and/or (ii) a measured C-reactive protein factor value, and/or (iii) a measured heart rate variability factor value. This embodiment variant has, inter alia, the same advantages as the previous one, however, allows to even improve further the technical accuracy, flexibility and transparency of the automated system.

In another embodiment variant, in case of defined threshold values are exceeded by at least one of the captured factor values, the parametrization of the risk shape pattern can e.g. be further extended by the input measures comprising (i) a measured waist circumference factor value, if a threshold value of one of the build factor values is exceeded, and/or (ii) a measured apolipoproteins factor value, if a threshold value of at least one of the lipid factor values is exceeded, and/or (iii) a measured relationship and/or relative diagnosis age of family history factor value, if a threshold value of at least one of the family history factor values is exceeded, and/or (iv) a factor value indicating certain diabetes sub-types, if a threshold value of at least one of the glucose metabolism factor values is exceeded, and/or (iv) a measured sport-driven physical activity measures value, if a threshold value of at least one of the physical activity factor values is exceeded, and/or (v) a measured activity intensity qualifier factor value, if a threshold value of at least one of the physical activity factor values is exceeded, and/or (vi) a measured binge drinking value, if a threshold value of a drinking factor value is exceeded, and/or (vii) measured factor values based on an additional popular screening questionnaires, if a threshold value indicating mental wellness is exceeded. The sport-driven physical activity measures can e.g. comprise factor values at least indicating cycling and/or swimming activity of the individual. This embodiment variant has, inter alia, the same advantages as the previous one, however, allows to even improve further the technical accuracy, flexibility and transparency of the automated system.

In still another embodiment variant, the system comprises a machine-based, automated simulation structure modelling and capturing interactive effects between any of the factors of the parametrization of the risk shape pattern, the factors providing the input measures to the system. The machine-based, automated simulation structure can e.g. be machine-learning based. This embodiment variant has, inter alia, the same advantages as the previous one, however, allows the system to be further optimized by reducing the used measuring parameters to the minimal needed number. Thus, this embodiment variant, inter alia, allows to optimize data processing time and efficiency, due to a minimized set of required input measuring values.

Finally, in an embodiment variant, a digital, cloud-based marketplace platform provides automated, risk underwriting and risk assessment for health and/or life risks by configuring, launching and processing of customized firs-tier and/or second-tier risk-transfer products for risk-exposed living individuals as first units and carriers/brokers as second units, wherein an automated risk-transfer placement is provided by the digital platform in a digital environment by a first online channel comprising a parameter-driven, rule-based underwriting process for creating or participating at risk-transfer structures by means of a pricing and underwriting engine, wherein an automated claim handling is provided by the platform by means of a claim triage and handling engine as a second online channel, and wherein an automated accounting is provided by the platform by a balance sheet provision and management engine and policy administration engine as a third online channel, in that the digital platform comprises the measuring system for measuring relative risk measurands of living individuals as the described resilience score and each digital service of the platform by a contribution measure to an extended resilience score of the living individuals purchasing the risk-transfer products on the digital marketplace platform and benefiting from the digital services of the digital platform, in that the extended resilience score provides a measure based on the measured current health status of the living individuals and/or the measured probability to purchase risk-transfer cover and/or the measured probability to start or keep behavior for improving their health status, in that the measuring of the extended resilience score encompasses different type of risks at least comprising mortality risk and/or morbidity risks and/or longevity risks together with the probability to claim for a risk-transfer benefit and/or the measured evolving health status of the living individuals, and in that the contribution measure to the extended resilience score is measured by assessing the variance of an individual's (2) extended resilience score by changing first individual's parameters at least comprising adding or omitting a specific risk-transfer cover and/or triggering start or maintenance of a nutrition program.

This embodiment variant has inter alia the advantage that it provides an automated electronic digital channel to place and manage risks and risk-transfers between first risk-transfer systems or insurers and second risk-transfer systems reinsurers, in particular a digital B2B and B2C channel. The invention provides the technical infrastructure as a digital marketplace for an automated one-stop system comprising automated underwriting and user-specific data capturing, automated claim handling, automated accounting (technical and financial) and automated reporting all in one technical system and a based on the same extended resilience score as risk-transfer quality indicator. The system is able to provide automated risk-transfer coverage for all risk-specific fields as e.g. property and casualty risks, life and health risks, any line of business or industry risks, single risks, treaty and facility risks, and accumulation or clash risk involving loss exposure of one event spreading to multiple lines of business, i.e. correlated risk structures. The inventive system allows in a new technical way a user to monitor and fully control his risks along the entire value chain at any time. By the inventive extended resilience score, the invention provides a new kind of direct and full control and transparency of the risk-transfer portfolio to the user, in particular the invention provides early recognition of trends and agile risk-transfer steering by means of forward- and backward-looking metrics and measure values. The invention also provides technical means and API's which can be seamless integration with other technical solutions and systems as e.g. portfolio monitoring platforms. Thus, B2B and B2C users will have specific benefits for participating in the digital marketplace; for instance, (A) B2B benefits (for insurers and other distributors) are (i) access to consumer pool that is cheaper (streamline costs e.g., reducing acquisition costs for consumers on the marketplace, sharing back-office tools/processes, sharing legal checks/regulatory requirements) or broader (extend product offering; expand to new geographies; sell to individuals not traditionally interested in insurance, priced out or excluded) vs today), (ii) validated assessment of current status of resilience for each individual consumer; (iii) validated assessment of impact of services in the individual's resilience; and (iv) access to more and curated roster of service providers; and (B) B2C benefits (for consumers) are: (i) global portability of (resilience-relevant) data—e.g., data currently used to underwrite individuals; (ii) global portability of insurance coverage and/or service provision across geographies or providers; and (iii) security of individual data stored in a neutral platform vs each distributor.

### BRIEF DESCRIPTION OF THE DRAWINGS

The present invention will be explained in more detail below relying on examples and with reference to these drawings in which:

FIGS. 1-4 shows a diagram illustrating schematically an exemplary structure of the inventive system and the different embodiment variants denoted as phase 1, phase 2 and phase 3, in particular (i) P2: Big Six Lifestyle model, and (ii) Bio-Markers and other specific developing ideas within P3. As “Big Six Lifestyle Model” (P1) is the narrowest embodiment variant. Further, also broader embodiment variants covered by phase 2 and phase 3.
(i) Phase 1: Big Six Lifestyle Model: (a) Embodiment variant of underwriting relative risk ratings for the “Big 6”; (b) The “Big 6” are: Physical activity, Sleep, Mental health, Nutrition, Substance, and Environment
(ii) Phase 2: (a) Further enhanced the “Big 6” research, (b) Full review of all clinical risk factors in our existing calculator (Life Guide's CVS calculator). This includes: Build (height and weight), Lipids (total cholesterol, HDL, triglycerides), Blood pressure (systolic and diastolic blood pressure), Glucose metabolism (fasting/non-fasting glucose, A1c, diabetes status), Liver function tests (GGT, ALT, AST, Alk Phos), and Family history of diabetes and circulatory disorders, (c) Added new clinical risk factors into the calculator. This includes: Calcium scores, C-reactive protein, and Heart rate variability, (d) Extended the input measures available under different risk factors. This includes: Waist circumference (Build), Apolipoproteins (Lipids), Relationship and Relative diagnosis age of family history (Family history), Certain diabetes sub-types (Glucose metabolism), Sport-driven physical activity measures e.g. participation in cycling or swimming (Physical activity), Activity intensity qualifier (Physical activity), Binge drinking (Alcohol), and Additional popular screening questionnaires (Mental wellness), (e) Modelling of the interactive effects between risk factors.
   This phase 2 capability will exist in our CVS Calculator in Life guide and be available in Magnum. This Calculator can be modified to produce relevant Consumer facing scores to match a specific development in a given market (Relative Risk Rating, Health Age etc.). We will have a PRS service offering that enables non-Magnum clients to access the risk assessment and scores What is Unique about the New CVS Calculator?: Whilst many consumer risk scores exist. These have all typically been built from outside of insurance and underwriting, and do not fully integrate to an insurers underwriting manual. This means that disconnects exist between the risk score, and the data/assessment required by an insurer if they are to fully embed into in their product to drive underwriting and pricing. Workarounds are required to plug the gaps between the consumer score on the one hand, and the insurers risk assessment on the other. (EG Adding additional underwriting questions, pricing gaps).
   The New Risk Calculator directly incorporates the new lifestyle risk factors as an integral aspect of our Life Guide Philosophy. The PRS is therefore uniquely built from within our underwriting assessment and can plug directly into an insurers underwriting and pricing.
   The result of the risk calculator assessment can be expressed as both a consumer facing score, as well as a traditional underwriting output ensuring transparency of result in a format that makes sense to all parties. In summary, the embodiment variant of phase 1 (Big Six Model) provides a unique connection to underwriting.
(iii) Phase 3: Wider project and activities: One aspect within the structure developed under P3 relates to engage multiple partners over time and build an ecosystem of modelling structures and risk scores on a platform within an overall “container” system. Other aspects are shown in the FIGS. 5-8.

FIG. 5 shows a block diagram, schematically illustrating the automated end-to-end process according to the invention providing an efficient, automated online marketplace platform comprising underwriting, claims and accounting channels for users. The reference number 7 denotes the automated end-to-end process, 71 the automated underwriting process by means of a rule-based bifurcation process, 711 the creation of a submission, 712 the receiving and binding of a quotation, 713 the modifying and renewing of an acceptances, 72 the technical accounting process, 721 the booking of the premiums, 722 the advising on new claims, 723 the booking and updating of claims, 724 the rectifying of premiums, 725 the submission of a statement of accounts, 73 the financial accounting process, 731 the advising and/or requesting of payments, 732 the seamless pairing, and finally 733 the setting of the accounts. The proposed invention and method provides a fast and easy access to first and secondary risk-transfer underwriting, technical and financial accounting on a digital marketplace platform as digital environment. The invention allows to reduce technical and administrative input and costs for managing risk-transfer portfolios in the field of L&H risk measurement/assessment and risk-transfer (including UW and claim handling), covering the whole process by providing a fully automated consumer (and insurer/carrier) platform. A core feature is given by the platform by providing automated risk assessment in combination with the measured resilience score.

FIG. 6 shows a block diagram illustrating schematically an exemplary structure of the digital marketplace platform. As such, the digital platform is based on the use of digital platform technology and cloud-based technologies to provide a digital environment of supplier (here carriers such as AXA etc.) and consumers (insured) that participates in transactions, centralized analysis and exchanges.

FIGS. 7 and 8 show a block diagram illustrating schematically an exemplary fully automated consumer (insured) and carrier/broker digital risk-transfer marketplace platform. The digital marketplace is centered on consumers and distributors along value chain. The platform dynamically provides offering that evolves with individual along lifecycle. The platform structure can be applied to any risks (LH/PC) and any channel (on/offline). The digital platform allows users to access and use the marketplace based on or without charging an appropriate fee. The platform enables and supports the users, aligning the users' interests to the extent possible, cutting out possible interference with users' decisions on product details, targeting, channels etc., underlying and/or commodity costs occur only once, and multiple, flexible add-ons are enabled. The digital platform enables full portability, i.e. consumer (first units) can “natively” move across providers, across geographies. It further provides and is based on a global personal Resilience Score measure, using multiple data sources. Each consumer's data is segregated and hold confidential and secure. Carriers can reinsure on reinsurer's balance sheet, however, don't have to. The platform provides further addons including: Access to (any) service provider via API, process automation for UW and claim handling, consumer advisory app, powered by the Resilience Score, and consumer-level insights, anonymized for benchmarking, pricing. The reference Magnum in the figures denotes a fully automated cloud-based digital solution for secure underwriting, individual risk assessment/measurement, and automated portfolio handling.

### DETAILED DESCRIPTION OF THE PREFERRED EMBODIMENTS

FIGS. 1 to 4 schematically illustrate an architecture for a possible implementation of an embodiment of the inventive, automated risk measuring and assessment system 16 for measuring relative risk measurands of living individuals 2. The relative risk measurands providing a measure for the frequency of occurrences of specific medical events having impacting consequences in specified ranges to the living individual 2 within a defined cohort of living individuals 2 relative to a randomized cohort of living individuals 2.

The system 16 comprises a defined parametrization for capturing risk shape pattern for living individual 2. The parametrization of the risk shape pattern comprises at least lifestyle factor values measuring physical activity and/or sleep and/or nutrition and/or mental wellbeing and/or substance use and/or environmental conditions. The parametrization of the risk shape pattern can e.g. further comprises clinical factor values measuring (i) build factor values comprising a measured height and/or weight factor value, and/or (ii) lipids factor values comprising a measured total cholesterol factor value and/or a high-density lipoprotein factor value and/or a triglycerides factor value, and/or (iii) blood pressure factor values comprising a measured systolic and diastolic blood pressure factor value, and/or (iv) glucose metabolism factor values comprising a measured fasting/non-fasting glucose factor value and/or glycated hemoglobin (hemoglobin A1c) factor value and/or diabetes status, and/or (v) liver function factor values comprising a measured gamma-glutamyltransferase (GGT) factor value and/or an alanine transaminase (ALT) factor value and/or an aspartate transaminase (AST) factor value and/or an alkaline phosphatase (Alk Phos), and/or (vi) family history of diabetes and circulatory disorders factor values. The parametrization of the risk shape pattern can e.g. further comprise clinical factor values measuring (i) a measured calcium score factor value, and/or (ii) a measured C-reactive protein factor value, and/or (iii) a measured heart rate variability factor value. In case of defined threshold values are exceeded by at least one of the captured factor values, the parametrization of the risk shape pattern is further extended by the input measures comprising (i) a measured waist circumference factor value, if a threshold value of one of the build factor values is exceeded, and/or (ii) a measured apolipoproteins factor value, if a threshold value of at least one of the lipid factor values is exceeded, and/or (iii) a measured relationship and/or relative diagnosis age of family history factor value, if a threshold value of at least one of the family history factor values is exceeded, and/or (iv) a factor value indicating certain diabetes sub-types, if a threshold value of at least one of the glucose metabolism factor values is exceeded, and/or (iv) a measured sport-driven physical activity measures value, if a threshold value of at least one of the physical activity factor values is exceeded, and/or (v) a measured activity intensity qualifier factor value, if a threshold value of at least one of the physical activity factor values is exceeded, and/or (vi) a measured binge drinking value, if a threshold value of a drinking factor value is exceeded, and/or (vii) measured factor values based on an additional popular screening questionnaires, if a threshold value indicating mental wellness is exceeded. The sport-driven physical activity measures comprise factor values at least indicating cycling and/or swimming activity of the individual 2.

The system 16 captures a multitude of risk shape pattern for living individuals 2 at least by means of said lifestyle factor values. The captured multitude of risk shape pattern are clustered by the system 16, each cluster defining a prototype of risk shape pattern assigned to a dedicated relative risk measurand.

A newly captured risk shape pattern of a living individual 2 is mapped by the system 16 to one of the prototypes of risk shape pattern based on measured lifestyle factor values associated with the living individual 2. The dedicated relative risk measurand assigned to the risk shape pattern is outputted as resilience score 161 value of the living individual 2.

As a variant, the system 16 can e.g. further comprise a machine-learning based simulation structure modelling and capturing interactive effects between any of the factors of the parametrization of the risk shape pattern, the factors providing the input measures to the system 16. The machine-learning structure can e.g. be applied to a digital representation (twin) of the living individual allowing a seriatim predictive processing of the measuring parameter values. The system 16 and inventive data processing can e.g. comprise a data pre-processing or data cleaning step where noisy data or outliers are removed from the target dataset. This step also encompasses a processing structure needed to deal with the inconsistencies in the target data. In case of discrepancies, those variables will be transformed to allow data processing and interpretation within the measuring parameters. In this step, the measuring parameter values and data is cleaned to treat missing values to make the data consistent with data processing. The data sets of the system may have attributes with a remarkable amount of missing data. The missing data structure and mechanism will be accomplished for the data set. For the present invention, there exist at least three mechanisms suitable for missing data completion, namely, Missing Completely At Random (MCAR), Missing At Random (MAR), and Missing Not At Random (MNAR). MCAR can be the case when the distribution of the missing values does not show any relationship between the observed data and the missing data. In other words, the missing values are like a random sample of all the cases in the feature. The MAR mechanism requires that the missingness may be dependent on other observed variables, but independent of any unobserved features. In other words, missing values do not depend on the missing data, yet can be predicted using the observed and or measured data. Finally, the MNAR mechanism is applied when the missing pattern relies on the unobserved variables; that is, the observed part of the data cannot explain the missing values. This missing data mechanism is the most difficult to treat as it renders the usual and normally used imputation methods meaningless.

Further, for the realization of the present invention, the system comprises a dimensionality reduction involving reducing the number of variables to be used for efficient modeling. The proposed structure can be broadly divided into feature selection and feature extraction. Feature selection is herein realized as a process involved in selecting the prominent variables, whereas the feature extraction applied to transform the high dimensional data into fewer dimensions to be used in building the models. Thus, dimensionality reduction is used to train the used machine learning structure faster and more efficient as well as increase model accuracy by reducing model overfitting.

For feature selection, one of the following techniques can e.g. be used, herein denoted as filter method, wrapper method, and embedded method. The filter method uses a ranking to provide scores to each variable, either based on univariate historical measuring parameters or depending on the target variable. The rankings can then be assessed to decide whether to keep or discard the variable from the analysis. The wrapper method, on the contrary, takes into account a subset of features and compares between different combinations of attributes to assign scores to the features. The embedded method is slightly more complicated to be realized in the context of the present invention, since the learning method usually decides which features are best for a model while the model is being built. Attributes can be selected based on Pearson's correlation, Chi-square, information gain ratio (IGR), or another appropriate technique. In contrary, the feature extraction process provides new features from the original features, to increase the accuracy via eliminating redundant features and irrelevant features. This research limits itself on two methods, namely the correlation-based feature selection method and principal component analysis-based feature extraction method.

Correlation-based feature selection is implemented to generate subsets of attributes in order to select a useful subset of features contains highly correlated features with the class, yet uncorrelated to each other. This feature selection processing is easy to understand and fast to execute. It removes noisy data and improves the performance of the used data processing structures. It does not require the system to put any limits on the selected number of attributes but generates the optimal number of features by itself. The correlation values for the feature selection are not only generated based on Pearson's correlation coefficient but are based on the measures namely, minimum description length (MDL), symmetrical uncertainty, and relief. CFS requires the nominal attributes in a data set to be discretized before generating the correlation. Nonetheless, for the present inventive system, it works on any data set, independent of the data transformation methods used. In fact, for the present system CFS was measured to be more accurate compared to IGR. Similarly, concluded that they obtained the highest accuracy for their classification using a CFS as compared to other feature selection methods.

Principal components analysis (PCA) can be used as an unsupervised linear feature extraction technique aimed at reducing the size of the data by extracting features having most information. PCA uses the features in the data set to create new features, known as the principal components. The principal components are then used as the new attributes to create the prediction model. The principal components have better explaining power compared to the single attributes. The explaining power can be measured by the explained variance ratio of the principal components. This value shows how much information is retained by the combined features. PCA works by generating eigenvalues of the correlation matrix of the attributes. The variance explained by each newly generated component is determined and the components retained are those which have the maximal variation in the data set. In fact, the PCA method is found to be more useful when used with the predictive structure of the present invention.

For the present invention, at least one of the following four machine learning algorithms can be implemented on CFS and PCA, namely, Multiple Linear Regression, REPTree, Random Tree, and Multilayer Perceptron. Multiple linear regression allows to provide the relationship between the response variable and at least two predictor variables by fitting a linear relation to the observed data points. In other words, the linear relation is used to predict the response variable based on the values of the explanatory variables collectively. Multiple linear regression structure can be implemented based on the generated sum of squared errors which shows the average distance of the predicted data points to the observed data values. The model parameter estimates can be generated to minimize the sum of squared errors by the inventive system, such that the accuracy of the modelling structure is increased. The variables significance in the regression relation can be determined by implementing statistical methods and are mostly based on the collinearity and partial correlation statistics of the explanatory features.

The REPTree classifier is a type of decision tree classification technique, which also can be used with the present invention. This method allows to automatically build both classification and regression trees, depending on the type of the response variable. A decision tree can be created in case of discrete response attribute, while a regression tree is developed if the response attribute is continuous. For the present invention, decision trees are a useful machine learning technique for classification problems. The decision tree structure is realized comprising of a root node, branches, and leaf nodes aimed at representing data in the form of a tree-like graph. Each internal node represents the tests performed, and the branches are representative of the outcome of the test. The leaf nodes, on the other hand, represent class labels. Decision trees mainly use the divide and conquer structure for prediction purposes. Thus, decision trees can be used as machine learning techniques for prediction and have been implemented for the present invention. The advantage of using decision trees is that they are very transparent in their present implementation. REPTree stands for Reduced Error Pruning Tree making use of regression tree logic to create numerous trees in different iterations. This structure is used as it is a fast learner, which develops decision trees based on the information gain and variance reduction. After creating several trees, the processing structure chooses the best tree using the lowest mean-square-error measure when pruning the trees.

The Random Tree is also a decision tree algorithm applicable for the present invention, however it is different from the previously disclosed application of the REPTree structure in the way it works. Random Tree, as machine learning algorithm, accounts for k randomly selected attributes at each node in the decision tree. In other words, random tree classifier builds a decision tree based on random selection of data as well as by randomly choosing attributes in the data set. Unlike REPTree classifier, this data processing structure performs no pruning of the tree. The structure works in a way that it conducts backfitting, which means that it estimates class probabilities based on a hold-out set. For the present inventive system, the random tree classifier can be used together with CFS allowing the classifier to work more efficiently with large data sets. Further, this embodiment variant allows to achieve high levels of modelling accuracy by modifying the parameters of the random tree classifier.

Finally, also artificial neural network structures can be applied. They comprise many highly interconnected processing elements, known as the neurons. For the present invention, the neurons can e.g. be organized in three layers, which are the input, hidden, and output layers. The neurons keep learning to improve the predictive performance of the structure used. This adaptive learning capability of the structure is very beneficial for providing high accuracy prediction given a data set for training. The neural network structure can e.g. use of backpropagation to classify instances. Backpropagation herein refers to a supervised learning structure which generates A the error of each neuron after a subset of the data is processed and distributes back the errors through the layers in the network. As an embodiment variant, the neural network structure can also be altered and/or adjusted when it is trained.

As illustrated by the FIGS. 1 to 4 and as described above, the present invention can e.g. be realized following one of the embodiment variants (i) “P2—Big Six Model”, (ii) “P3—PRS” (alternative data as digital biomarkers) and (iii) “P3—Enrich” (insights generation using machine learning/predictive modelling, natural language processing etc.), inter alia, providing the technical risk assessment basis for a fully automated, global, digital, resilience marketplace 1. The three embodiment variants P2—Big Six Model, P3—PRS and P3—Enrich are based on a Personal Resilience Score (PRS)-based digital marketplace and digital platform providing services for applicants/consumers and distributors (carriers/first insurers) along the complete value chain for any risk exposure, i.e. L&H (Life and Health) risk-transfer (insurance) based on a Personal Resilience Score (PRS) index measure. In particular, the invention provides L&H risk measurement, assessment and risk-transfer (including all process steps from UW to claim handling), covering the whole process by providing a fully automated consumer and insurer/carrier platform. Key features of the inventive system are the central data capturing of all relevant data dimension in combination with a defined, reproducible index score measure.

The embodiment variant P2—Big Six Model incorporates key clinical risk factors and lifestyle factors using a “Big Six Lifestyle Modeling” structure to parametrize the Personal Resilience Score (PRS) measure. The six lifestyle factors are given by (i) Physical activity; (ii) Sleep; (iii) Mental health; (iv) Nutrition; (v) Substance use; and (vi) Environment. The key clinical risk factors include build (height and weight), lipids (total cholesterol, HDL, triglycerides), blood pressure (systolic and diastolic blood pressure), glucose metabolism (fasting/non-fasting glucose, A1c, diabetes status), liver function tests (GGT, ALT, AST, Alk Phos), and family history of diabetes, circulatory disorders, calcium scores, C-reactive protein, and heart rate variability. P2—Big Six Model further extends the input measures available under different risk factors and uses additional modelling of the interactive effects between risk factors.

The second embodiment variant “P3—PRS” is based on a further extended modelling structure using additional alternative data, such as digital biomarkers, financial data and consumer data. Finally, the third embodiment variant “P3—Enrich”, finally, provides insights generation for a user using machine learning/predictive modelling, natural language processing etc.

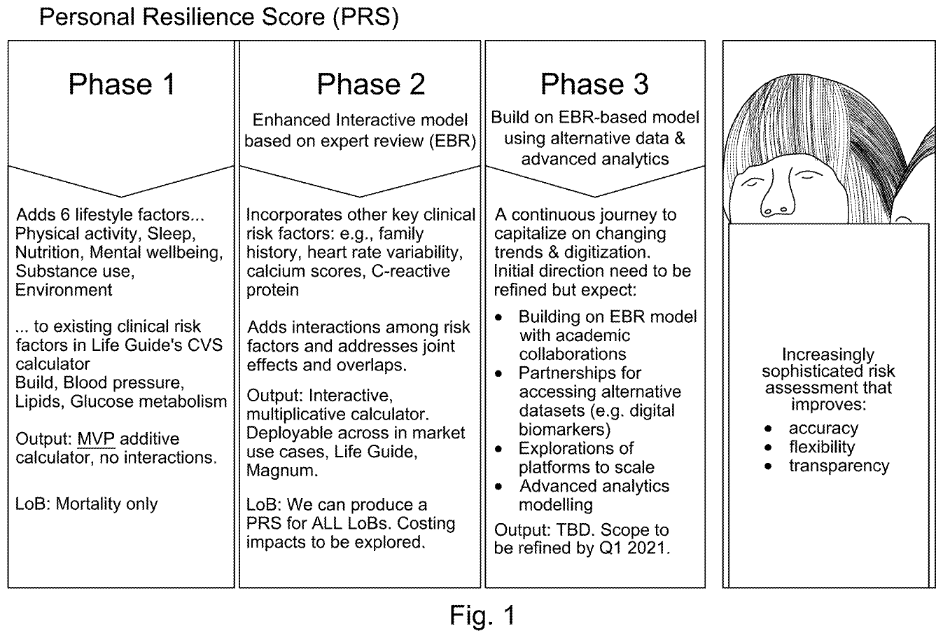

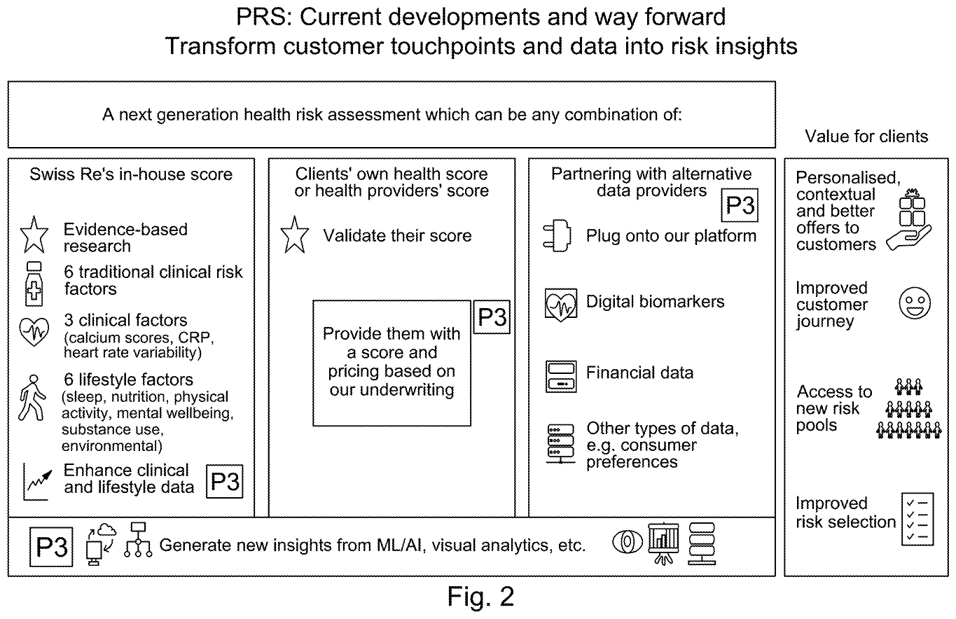

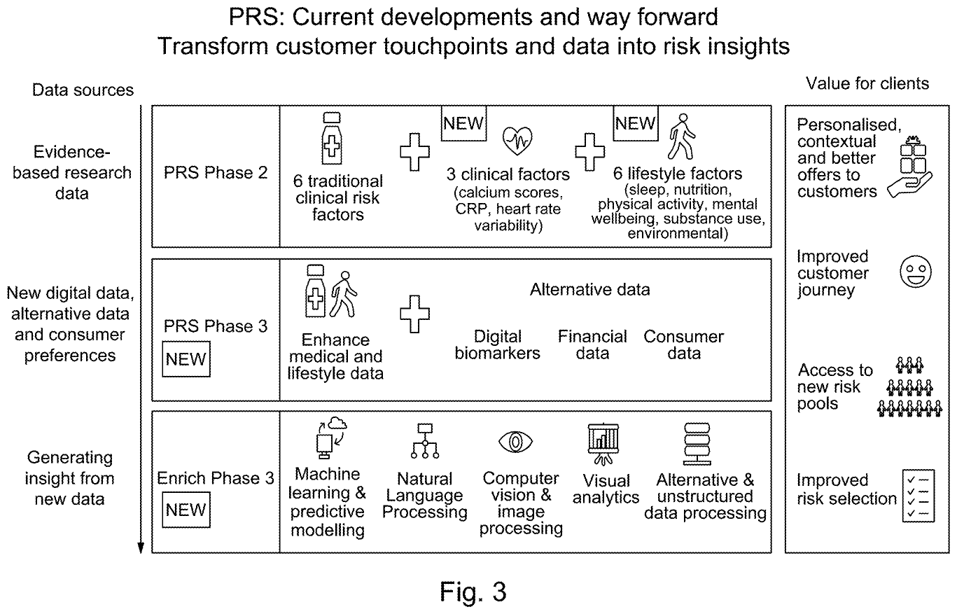

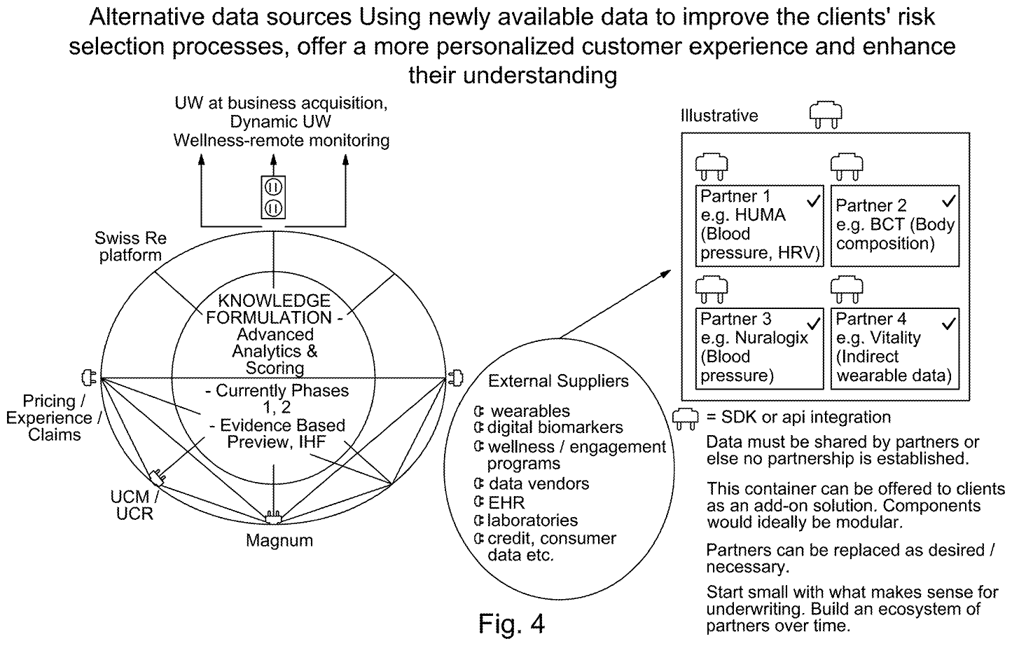

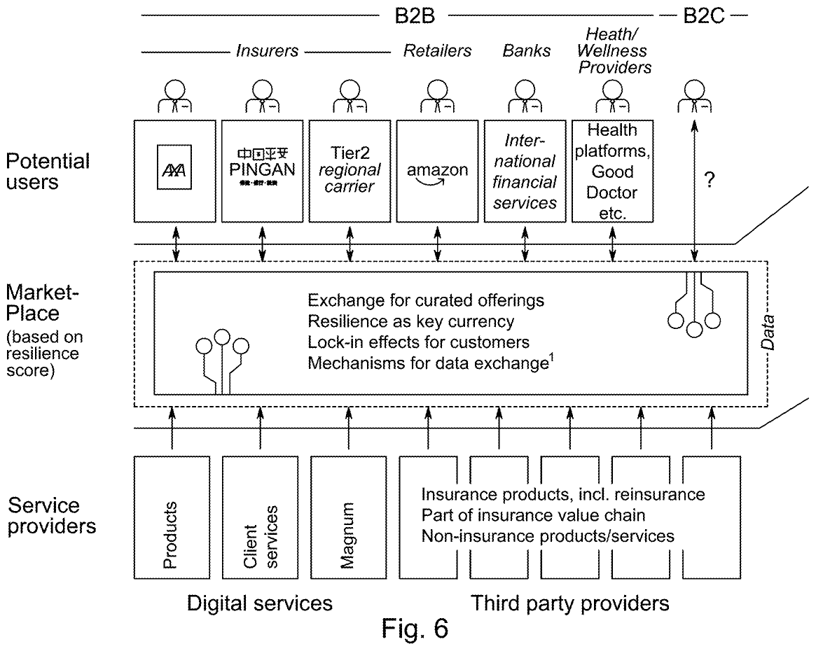

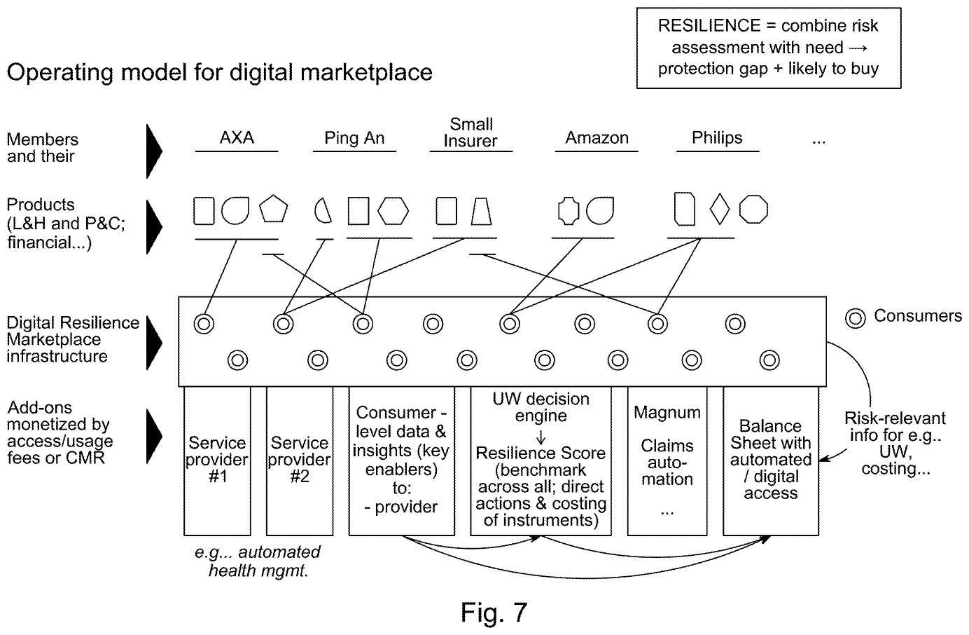

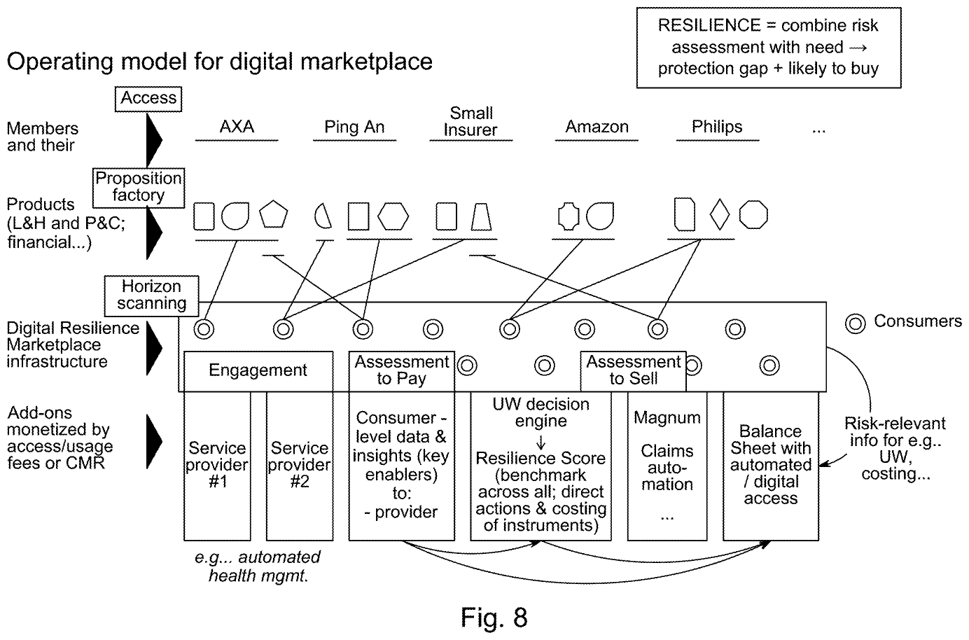

The inventive, automated risk measuring and assessment system 16 for measuring relative risk measurands of living individuals 2 can form the technical basis and/or integrated technical part of a digital platform marketplace 1. FIGS. 5 to 8 schematically illustrate such an architecture for a possible implementation of the inventive system 16 in a digital system and platform 1 respectively digital platform 1 allowing to compose, launch and configuration of first and second-tier risk-transfer with built-in auditing and monitoring structures based on a resilience score measure. In the present invention, the quantitative extended resilience score 1611 is provided as an outcome of the B2B/B2C users participating in the marketplace 1, where the extended resilience is a quantitative measure for the generic operational benefit. Thus, the term “resilience marketplace” is a digital marketplace that thanks to the interaction of B2B/B2C users and the services providers give at the digital market place, creates more “personal resilience” for the consumers that end up buying the insurance products.

As FIG. 5 shows, the inventive, automated end-to-end process provides an efficient, automated online risk placement, claims and accounting channel for users with a complete electronic solution for automated business structuring. Thus, this embodiment variant provides, in the field of L&H risk-transfer, a digital marketplace platform 1 with automated risk assessment system 16 and automated risk-transfer (including UW and claim handling), covering the whole process by providing a fully automated consumer (and insurer/carrier) platform. A key feature is given by the automated risk assessment system 16 in combination with the resilience score 161.

The digital platform 1 provides automated risk assessment 16 in combination with the resilience score 161 and the extended resilience score 1611, where the overall solution consists of curated offering for B2B and potentially B2C “users” or marketplace members: insurers, other distributors of insurance and other financial products, and direct consumers. Each will have a specific benefit for participating in the digital marketplace; for instance, (A) B2B benefits (for insurers and other distributors) are (i) access to consumer pool that is cheaper (streamline costs e.g., reducing acquisition costs for consumers on the marketplace, sharing back-office tools/processes, sharing legal checks/regulatory requirements) or broader (extend product offering; expand to new geographies; sell to individuals not traditionally interested in insurance, priced out or excluded) vs today), (ii) validated assessment of current status of resilience for each individual consumer; (iii) validated assessment of impact of services in the individual's resilience; and (iv) access to more and curated roster of service providers; and (B) B2C benefits (for consumers) are: (i) global portability of (resilience-relevant) data—e.g., data currently used to underwrite individuals; (ii) global portability of insurance coverage and/or service provision across geographies or providers; and (iii) security of individual data stored in a neutral platform vs each distributor.

In the digital marketplace platform 1, each individual and service provided on the digital marketplace is automatically rated on its contribution to an extended resilience measure 161 of the individuals that purchase products on the digital marketplace and benefits from its services. The extended resilience score measure rates the current health status of individuals, and their likelihood to purchase insurance protection or start/keep behaviors that improve their health status. Current means that it can dynamically change (and be reassessed and recalculated) over the duration of the policy, i.e. the term of the risk-transfer, due to changes in health status, behaviors, illnesses . . . .

As such, the digital, cloud-based marketplace platform 1 provides fully automated, end-to-end risk-assessment and risk-transfer processes by configuring, launching and processing of customized firs-tier and/or second-tier risk-transfer products 4/41, 42, . . . , 4i for risk-exposed individuals as first units 2 and carriers/brokers as second units 3. An automated risk-transfer placement 7 is provided by the digital platform 1 in a digital environment by a first online channel comprising a parameter-driven, rule-based underwriting process 71 for creating or participating at risk-transfer structures by means of a pricing and underwriting engine 13. An automated claim handling 72 is provided by the system 1 by means of a claim triage and handling engine 14 as a second online channel, and an automated accounting 73 is provided by the platform 1 by a balance sheet provision and management engine and policy administration engine 15 as a third online channel. The digital platform 1 comprises the inventive risk assessment and measuring system or engine 16 for rating each individual 2 and each digital service 181, 182, . . . , 18i of the platform 1, e.g. provided by the monitoring and surveillance unit 18, by a contribution measure to an extended resilience score 1611 of the first units 2 purchasing the risk-transfer products 4/41, . . . , 4i on the digital marketplace platform 1 and benefiting from the digital services 181, 182, . . . , 18i of the digital platform 1. The extended resilience score 161 provides a measure based on the measured current health status 162 of the first unit 2 and/or the measured probability to purchase risk-transfer cover 163 and/or the measured probability to start or keep behavior for improving their health status 164. The measuring of the extended resilience score 1611 encompasses different type of risks at least comprising mortality risks 1651 and/or morbidity risks 1652 and/or longevity risks 1653 together with the probability to claim for a risk-transfer benefit (166) and/or the measured evolving health status 162 of the first unit 2. Finally, the contribution measure to the extended resilience score 1611 is measured by assessing the variance of an individual's resilience score 161 by changing first unit's parameter at least comprising adding or omitting a specific risk-transfer cover and/or triggering start or maintenance of a nutrition program.

It is to be noted that digital marketplaces denote specialized digital platforms and database structures which allow to connect consumers and carriers (B2C) and carriers and carriers (B2B) typically requiring cross-network interaction and collaboration, where a large number of data exchanges amongst insurer and customers' needs to be performed. However, proprietary platforms often cannot ensure to be fully aligned of the customer's interests, in particular having no interference with customers' decisions on product details, targeting, channels etc. On the other hand, such proprietary platforms do not provide the desired portability to consumers, so that they can “natively” move across providers/carriers and across geographies. One of the underlying problems is the lack of standardization as a driving factor for complexity and process frictions, particularly in (i) policy issuance, (ii) cash movement, (iii) risk engineering services, and (iv) claims handling.

Detected changes in the resilience score 161 or the extended resilience score 1611 can e.g. trigger changes in pricing parameters and/or benefit parameters of one or more risk-transfer or financial products. The digital marketplace platform 1 can e.g. comprises standardized and dedicated interfaces for registering and/or authentication and/or authorization of new or existing first or second units. The digital marketplace platform 1 can e.g. comprise standardized and dedicated interfaces for the integration of new second units as service providers on the digital marketplace platform 1. The digital marketplace platform 1 can also comprise standardized and dedicated application programming interface (API) for data exchange and transfer between the digital marketplace platform 1 and a second unit.

As an embodiment variant, the digital marketplace platform 1 can e.g. comprises dedicated electronic links to external digital insurance engine and/or automated underwriting capabilities and/or claim automation means. The digital marketplace platform 1 can comprise interfaces providing access to electronic, second tier (reinsurance) balance sheets. The electronic balance sheet can e.g. be a life and health risk-transfer balance sheet or a property and casualty risk-transfer balance sheet.

The digital services of the marketplace platform 1 comprise B2B for second units and/or B2C digital services between first and second units: The B2B digital services for second units at least can e.g. comprise an automated pricing and underwriting process by means of a pricing/underwriting engine. The B2B digital services can at least comprise an automated balance sheet provision and management engine providing automated accounting for second units, wherein a provision is represented by a digital account recording liabilities of a second unit arising from conducted risk-transfers, and wherein measurement of the amount assessed as provision is realized by best estimate of the expenditure required to settle the current detected obligation at a balance sheet date. The recording of the liability in a second unit's balance sheet can e.g. automatically matched to an assigned expense account. The B2B digital services at least can comprise an automated claim triage process and claim handling process processed by means of a claims triage and handling engine for second units. The B2B digital services can at least comprise an automated policy administration process processed by means of a policy administration engine between second units. The B2C digital services can further at least comprise an automated engagement and sales process accessible via the digital marketplace platform or via a client application accessing the digital marketplace platform provided by an automated engagement and sales engine.

The B2C digital services can at least comprise an allocation of automatedly adapted generic or user-specific programs, as dynamically adapted “health improvement/behavioral changes” programs, such as nutrition program processes, by means of a program engine. The adaption comprises an automated, dynamic assessment and measurement of the changing risks under the program process by the digital platform 1′. The B2C digital services can at least comprise an allocation of automatedly adapted health and disease management program processes by a health and disease management engine. The B2C digital services can at least comprise monitoring means for monitoring a current resilience status based on the measured resilience score accessible via an advisory application of the digital platform. The advisory application can e.g. comprise an expert module providing expert advices and opportunities for improvement of the individual resilience score.

The contribution to resilience of each component of the marketplace services can e.g. be assessed individually by the digital marketplace platform 1. The digital platform can e.g. comprise means for selecting and/or rating of each first and/or second unit. The digital platform can e.g. further comprise rating means for measuring and ensuring quality assurance.

The digital platform 1 can e.g. comprise means for assessing risk parameters capturing risk-exposure of first units as risk-exposed consumers (the term “consumers” denoting individuals exposed to L&H risks), wherein the digital platform can comprise a product configurator with a machine-based exposure data intelligence enabled to automatically identify risks of first units based on the captured risk-parameters of the first unit. The digital platform 1 can comprise a metric simulation engine for automated prediction of forward- and backward-looking impact measures based on risk-event parameter values of time-dependent series of occurrences of physically impacting risk-events, wherein the occurrences of the physical risk-events are measured based on predefined threshold-values of the event parameters and wherein the impacts of the risk-events to a specific first unit are measured based on impact parameters associated with the first unit. Finally, the digital platform 1 can e.g. comprise a graphical user interface of a portfolio analytics framework providing a dynamic representation of a portfolio structure, wherein a metric simulation engine forms an integrated part of the portfolio analytics framework, and wherein, by means of the metric simulation engine, the dynamic representation of the portfolio structure provides forward- and backward-looking insights to the user based on the measured resilience score thereby enabling portfolio steering by identification of critical areas of the portfolio and impacts of possible changes to the underwriting.

### LIST OF REFERENCE SIGNS

1 Digital Marketplace
1′ Digital Marketplace Platform
10 Secure Data Transmission Network Interface
11 Marketplace Module
12 Persistent Storage
121 Data Segment comprising risk-transfer data elements 121i
1211, 1212, . . . , 121i Digital risk-transfer Data Element
122 Data Segment comprising consumer data elements 122i
1221, 1222, . . . , 122i Digital Consumer Data Element
123 Data Segment comprising carrier/broker data elements 123i
1231, 1232, . . . , 123i Digital Carrier/Broker Data Element
13 Pricing and underwriting engine
14 Claim triage and handling engine
15 Accounting module/Balance sheet provision and management/Policy administration engine
16 Resilience Score Rating Module
161 Resilience score
1611 Extended resilience score
162 Health status (current/evolving)
163 Probability to purchase risk-transfer cover
164 Probability to start or keep behavior for improving their health status
165 Risks encompassed by the resilience score
1651 Mortality risks
1652 Morbidity risks
1653 Longevity risks
166 Probability to claim for a risk-transfer benefit
17 Web Server/Network Server
171 Firewall
172 Router
18 Monitoring and surveillance unit
181 Monitoring of the contribution to resilience of each component of the marketplace services
182 Selection/rating of each provider monitoring
183 Quality assurance monitoring
184 Risk engineering parameter monitoring
185 Expert system advising with knowledge and parameters insights
2 Living individual (first units)
21 Individual Unit (C₂₁) associated with consumer unit data element 1221
22 Individual Unit (C₂₂) associated with consumer unit data element 1222
2i Individual Unit (C₂₁) associated with digital consumer unit data element 122i
2i11-2i1x Sensory and measuring devices of the digital platform 1 measuring and capturing of measuring data measuring risk-exposer and characteristics of consumer unit 2i by means of the sensory and measuring devices associated with the unit 2
3 Carrier/broker units (second units)
31 Carrier/Broker Unit (B₂₁) associated with carrier/broker unit data element 1231
32 Carrier/Broker Unit (B₂₂) associated with carrier/broker unit data element 1232
3i Carrier/Broker Unit (B₂₁) associated with carrier/broker unit data element 123i
4 Risk-transfers
41 Risk-transfer (R₄₁) associated with the risk-transfer data element 1211
42 Risk-transfer (R₄₂) associated with risk-transfer data element 1212
4i Risk-transfer (R₄₁) associated with risk-transfer data element 121i
5 Secured Network and Network Accesses to digital marketplace platform
6 Data transmission Network
61 Internet, Worldwide Backbone Network
7 Processes
71 Automated parameter-driven, rule-based underwriting process
771 Rule-based bifurcation process
772 Receiving and binding of a quotation
773 Modifying and renewing of an acceptances
72 Automated claim handling processes
721 Booking of premiums
722 Advising on new claims
723 Booking and updating of claims
724 Rectifying of premiums
725 Submission of a statement of accounts
73 Automated accounting processes
731 Advising and/or requesting of payments
732 Seamless pairing
733 Setting of the accounts
8 3rd-party service provider units providing services as (i) health management/behavior programs (nutrition etc.) and/or (ii) other risk scoring/risk assessment (e.g. credit scoring). Units 8 are connected to the consumer data 122 but sit outside the digital market place 1′ and can provide additional inputs to the resilience score rating module 16.
81 Health management/behavior programs (nutrition etc.)
82 Other risk scoring/risk assessment (e.g. credit scoring)

1. A measuring system for measuring relative occurrence frequencies or measurable occurrence probabilities of a medical event and/or health event and/or life risk event associated with living individuals in a measurement future time window as relative risk measurand values based on measured physical parameter values by sensory and measuring devices, wherein the relative risk measurands providing a measure for the frequency of physical occurrences of specific medical events and/or health events and/or life risk events having impacting consequences in specified ranges to the living individual within a defined cohort of living individuals relative to a randomized cohort of living individuals, the measuring system comprising:
processing circuitry configured to
perform a defined parametrization for capturing risk shape pattern for living individual, the parametrization of the risk shape pattern comprising at least lifestyle factor values measuring physical activity and/or sleep and/or nutrition and/or mental wellbeing and/or substance use and/or environmental conditions, wherein the risk shape pattern further comprises clinical factor values measuring (i) build factor values comprising a measured height and/or weight factor value, and/or (ii) lipids factor values comprising a measured total cholesterol factor value and/or a high-density lipoprotein factor value and/or a triglycerides factor value, and/or (iii) blood pressure factor values comprising a measured systolic and diastolic blood pressure factor value, and/or (iv) glucose metabolism factor values comprising a measured fasting/non-fasting glucose factor value and/or glycated hemoglobin (hemoglobin A1c) factor value and/or diabetes status, and/or (v) liver function factor values comprising a measured gamma-glutamyl-transferase (GGT) factor value and/or an alanine transaminase (ALT) factor value and/or an aspartate transaminase (AST) factor value and/or an alkaline phosphatase (Alk Phos), and/or (vi) family history of diabetes and circulatory disorders factor values,
perform a dimensionality reduction for reducing the used number of parameters by an integrated feature selection and feature extraction structure, wherein the system comprises a correlation-based feature selection structure for generating subsets of attributes by selecting a subset of features containing highly correlated features with a class of features, but uncorrelated to each other based on generated correlation values comprising at least Pearson's correlation coefficient, minimum description length (MDL), symmetrical uncertainty, and relief, and wherein the system comprises a principal components analysis (PCA) structure for unsupervised linear feature extraction reducing the size of the data by extracting features having most information,
capture a multitude of risk shape pattern for living individuals at least by said lifestyle factor values, wherein the captured multitude of risk shape pattern are clustered by the system, each cluster defining a prototype of risk shape pattern assigned to a dedicated relative risk measurand,
wherein a newly captured risk shape pattern of a living individual is mapped by the system to one of the prototypes of risk shape pattern based on measured lifestyle factor values associated with the living individual, and wherein the dedicated relative risk measurand assigned to the risk shape pattern is outputted as resilience score value of the living individual.

2. The measuring system for measuring relative risk measurands of a living individual according to claim 1, wherein the parametrization of the risk shape pattern further comprises clinical factor values measuring (i) a measured calcium score factor value, and/or (ii) a measured C-reactive protein factor value, and/or (iii) a measured heart rate variability factor value.

3. The measuring system for measuring relative risk measurands of a living individual according to claim 1, wherein, in case of defined threshold values are exceeded by at least one of the captured factor values, the parametrization of the risk shape pattern is further extended by the input measures comprising (i) a measured waist circumference factor value, if a threshold value of one of the build factor values is exceeded, and/or (ii) a measured apolipoproteins factor value, if a threshold value of at least one of the lipid factor values is exceeded, and/or (iii) a measured relationship and/or relative diagnosis age of family history factor value, if a threshold value of at least one of the family history factor values is exceeded, and/or (iv) a factor value indicating certain diabetes sub-types, if a threshold value of at least one of the glucose metabolism factor values is exceeded, and/or (iv) a measured sport-driven physical activity measures value, if a threshold value of at least one of the physical activity factor values is exceeded, and/or (v) a measured activity intensity qualifier factor value, if a threshold value of at least one of the physical activity factor values is exceeded, and/or (vi) a measured binge drinking value, if a threshold value of a drinking factor value is exceeded, and/or (vii) measured factor values based on an additional popular screening questionnaires, if a threshold value indicating mental wellness is exceeded.

4. The measuring system for measuring relative risk measurands of a living individual according to claim 3, wherein the sport-driven physical activity measures comprise factor values at least indicating cycling and/or swimming activity of the individual.

5. The measuring system for measuring relative risk measurands of a living individual according to claim 1, wherein the system comprises a machine-learning based simulation structure modelling and capturing interactive effects between any of the factors of the parametrization of the risk shape pattern, wherein the factors provide the input measures to the system.

6. A digital, cloud-based marketplace platform providing automated, risk underwriting and risk assessment for health and/or life risks by configuring, launching and processing of customized firs-tier and/or second-tier risk-transfer products for risk-exposed living individuals as first units and carriers/brokers as second units, wherein an automated risk-transfer placement is provided by the digital platform in a digital environment by a first online channel comprising a parameter-driven, rule-based underwriting process for creating or participating at risk-transfer structures by a pricing and underwriting engine, wherein an automated claim handling is provided by the platform by a claim triage and handling engine as a second online channel, and wherein an automated accounting is provided by the platform by a balance sheet provision and management engine and policy administration engine as a third online channel, wherein the digital platform comprises the measuring system for measuring relative risk measurands of living individuals based on physical parameter values measured by sensory and measuring devices as resilience score according to claim 1 and each digital service of the platform by a contribution measure to an extended resilience score of the living individuals purchasing the risk-transfer products on the digital marketplace platform and benefiting from the digital services of the digital platform,
wherein the platform comprises a dimensionality reduction for reducing the used number of parameters by an integrated feature selection and feature extraction structure, wherein the platform comprises a correlation-based feature selection structure for generating subsets of attributes by selecting a subset of features containing highly correlated features with a class of features, but uncorrelated to each other based on generated correlation values comprising at least Pearson's correlation coefficient, minimum description length (MDL), symmetrical uncertainty, and relief, and wherein the platform comprises a principal components analysis (PCA) structure for unsupervised linear feature extraction reducing the size of the data by extracting features having most information,
wherein the extended resilience score provides a measure based on the measured current health status of the living individuals and/or the measured probability to purchase risk-transfer cover and/or the measured probability to start or keep behavior for improving their health status,
wherein the measuring of the extended resilience score encompasses different type of risks at least comprising mortality risks and/or morbidity risks and/or longevity risks together with the probability to claim for a risk-transfer benefit and/or the measured evolving health status of the living individuals, and
wherein the contribution measure to the extended resilience score is measured by assessing the variance of an individual's extended resilience score by changing first individual's parameters at least comprising adding or omitting a specific risk-transfer cover and/or triggering start or maintenance of a nutrition program.

7. The digital, cloud-based marketplace platform according to claim 6, wherein detected changes in the extended resilience score triggers dynamically changes in pricing parameters and/or benefit parameters of one or more risk-transfer or financial products.

8. The digital, cloud-based marketplace platform according to claim 7, wherein dynamically detected changes in the resilience score trigger at least dynamic assessment and/or reassessment and/or repricing by the digital platform at inception and throughout a duration of a risk-transfer and/or of the user relationship established by the digital platform.

9. The digital, cloud-based marketplace platform according to claim 7, wherein in case a user has multiple risk-transfer products with multiple carriers/brokers, the digital marketplace uses same inputs to update the extended resilience score of the user which is then fed back to all carriers/brokers for dynamic reassessment and/or repricing of their respective risk-transfer products.

10. The digital, cloud-based marketplace platform according to claim 6, wherein the digital marketplace platform comprises standardized and dedicated interfaces for registering and/or authentication and/or authorization of new or existing first or second units.

11. The digital, cloud-based marketplace platform according to claim 6, wherein the digital marketplace platform comprises standardized and dedicated interfaces for the integration of new second units as service providers on the digital marketplace platform.

12. The digital, cloud-based marketplace platform according to claim 6, wherein the digital marketplace platform comprises standardized and dedicated application programming interface for data exchange and transfer between the digital marketplace platform and a second unit.

13. The digital, cloud-based marketplace platform according to claim 7, wherein the digital marketplace platform comprises dedicated electronic links to external digital insurance engine and/or automated underwriting capabilities and/or claim automation devices and/or external service providers of health or risk management program processes and/or automated risk assessments.

14. The digital, cloud-based marketplace platform according to claim 13, wherein the automated risk assessments at least comprise automated credit scores and/or credit scoring.

15. The digital, cloud-based marketplace platform according to claim 6, wherein the digital marketplace platform comprises interfaces providing access to electronic, second tier (reinsurance) balance sheets.

16. The digital, cloud-based marketplace platform according to claim 15, wherein the electronic balance sheet is a life and health risk-transfer balance sheet or a property and casualty risk-transfer balance sheet.

17. The digital, cloud-based marketplace platform according to claim 6, wherein the digital services of the marketplace platform comprise business-to-business (B2B) for second units and/or business-to-consumer (B2C) digital services between first and second units and/or B2B or B2C between users of the digital platform and external service providers of health or risk management program processes and/or automated risk assessments.

18. The digital, cloud-based marketplace platform according to claim 17, wherein the B2B digital services for second units at least comprise an automated pricing and underwriting process by a pricing/underwriting engine.

19. The digital, cloud-based marketplace platform according claim 17, wherein the B2B digital services at least comprise an automated balance sheet provision and management engine providing automated accounting for second units, wherein a provision is represented by a digital account recording liabilities of a second unit arising from conducted risk-transfers, and wherein measurement of the amount assessed as provision is realized by best estimate of the expenditure required to settle the current detected obligation at a balance sheet date.

20. The digital, cloud-based marketplace platform according to claim 19, wherein the recording of the liability in a second unit's balance sheet is automatically matched to an assigned expense account.

21. The digital, cloud-based marketplace platform according to claim 17, wherein the B2B digital services at least comprise an automated claim triage process and claim handling process processed by a claims triage and handling engine for second units.

22. The digital, cloud-based marketplace platform according to claim 17, wherein the B2B digital services at least comprise an automated policy administration process processed by a policy administration engine between second units.

23. The digital, cloud-based marketplace platform according to claim 17, wherein the B2C digital services at least comprise an automated engagement and sales process accessible via the digital marketplace platform or via a client application accessing the digital marketplace platform provided by an automated engagement and sales engine.

24. The digital, cloud-based marketplace platform according to claim 17, wherein the B2C digital services at least comprise an allocation of automatedly adapted nutrition program processes by a nutrition program engine.

25. The digital, cloud-based marketplace platform according to claim 17, wherein the B2C digital services at least comprise an allocation of automatedly adapted health and disease management program processes by a health and disease management engine.

26. The digital, cloud-based marketplace platform according to claim 17, wherein the B2C digital services at least comprise a monitoring device for monitoring a current resilience status based on the measured extended resilience score accessible via an advisory application of the digital platform.

27. The digital, cloud-based marketplace platform according to claim 26, wherein the advisory application comprises an expert module providing expert advices and opportunities for improvement of the individual resilience score.

28. The digital, cloud-based marketplace platform according to claim 6, wherein the contribution to resilience of each component of the marketplace services is assessed individually by the digital marketplace platform based on the consumers health status and/or the asset's risk characteristics.

29. The digital, cloud-based marketplace platform according to claim 6, wherein the digital platform comprises processing circuitry for selecting and/or rating of each first and/or second unit.

30. The digital, cloud-based marketplace platform according to claim 6, wherein the digital platform comprises processing circuitry for measuring and ensuring quality assurance.

31. The digital, cloud-based marketplace platform according to claim 6, wherein the digital platform comprises the digital platform comprises processing circuitry for assessing risk parameters capturing risk-exposure of first units as risk-exposed consumers, wherein the digital platform comprises a product configurator with a machine-based exposure data intelligence enabled to automatically identify risks of first units based on the captured risk-parameters of the first unit.
